(19) United States
(12) Patent Application Publication
(10) Pub. No.: US 2025/0213744 A1
(43) Pub. Date: Jul. 3, 2025

(54) ANTIMICROBIAL HYDROGEL COMPOSITION

(71) Applicant: UNIVERSITY OF BATH, Bath (GB)

(72) Inventors: Andrew Tobias Aveling JENKINS, Bath (GB); Robert David SHORT, Sheffield (GB)

(21) Appl. No.: 18/851,189

(22) PCT Filed: Mar. 27, 2023
(86) PCT No.: PCT/GB2023/050783

§ 371 Date: Sep. 26, 2024

(30) Foreign Application Priority Data
Mar. 28, 2022 (GB) 2204326.9

(51) Int. Cl. A61L 15/44; A61L 15/24; A61L 15/42

(52) U.S. Cl. CPC A61L 15/44; A61L 15/24; A61L 15/42; A61L 2300/104; A61L 2300/208; A61L 2300/23; A61L 2300/25; A61L 2300/404; A61L 2300/406; A61L 2300/602

(57) ABSTRACT

The present invention provides a composite hydrogel comprising: (a) an anionic or cationic functional group-containing polymer; and (b) at least one active pharmaceutical ingredient (API), wherein the API is oppositely charged to the anionic or cationic functional group-containing polymer. Methods of displacing an active pharmaceutical ingredient from the composite hydrogel comprising a step of treating the composite hydrogel with an ion source comprising anions, cations, or a combination thereof, a plasma jet consisting of excited gas, ions, electrons and photons, wound dressings comprising the composite hydrogel and methods of treating wounds using the composite hydrogel are also provided.

### FIELD OF INVENTION

The present invention relates to composite hydrogels comprising at least one active pharmaceutical ingredient that can be selectively released from the hydrogel for delivery to a patient.

### BACKGROUND TO THE INVENTION

Microbial infection is one of the principal aetiological factors in a wound becoming chronic (not healing within 6 weeks). The cost of caring for patients with a chronic wound is enormous: it is estimated to cost the UK National Health Service around GBP 3.2 billion/year to care for the 1.3 million chronic wound patients in the UK (2012/13 data).

It is estimated that the great majority of wound bacteria will be present as microbial communities referred to as biofilms. Biofilms are significantly harder to treat than their free-living counter parts and increase the risk of wound chronicity. Bacteria in the biofilm state are hard to remove by conventional antisepsis, often requiring 10-1000 times the minimum inhibitory concentration (MIC). Moreover, bacteria can secrete enzymes such as protease, hyaluronidase and elastase which break down dermal tissue providing a nutrient supply for the bacteria and deepening the wound; urease which raises local pH promoting further bacterial growth; and cytotoxins which cause direct cell damage.

Typically, chronic wounds are cared for by regular tissue debridement, deep cleaning, topical antiseptics and, if necessary, systemic antibiotics. However, debriding a wound also removes some healthy tissue and ultimately slows healing. Debridement is also often painful for the patient. In addition, the multicellular nature of biofilms can lead to antibiotic resistance, making the wound more difficult to treat.

Therefore there is a need to develop wound treatments which are more effective in treating chronic wounds and/or which allow wounds to heal without infection and scarring.

### SUMMARY OF THE INVENTION

According to a first aspect of the invention, there is provided a composite hydrogel comprising an anionic or cationic functional group-containing polymer and at least one active pharmaceutical agent (API), wherein the API is oppositely charged to the anionic or cationic functional group-containing polymer.

One challenge in designing hydrogels for delivery of APIs to skin, wounds or other topical areas is to trigger drug release following a specific stimulus. It is undesirable to have slow passive release from a hydrogel dressing if the drug being eluted is present in concentrations below its therapeutic limit. The composite hydrogel described herein allows at least one API to be delivered to skin, wounds or other topical areas under specific conditions in a dose-controllable and reproducible manner.

A second aspect of the invention provides a method of displacing an active pharmaceutical ingredient from a composite hydrogel according to the first aspect of the invention, the method comprising a step of treating the composite hydrogel with an ion source.

A third aspect of the invention provides a wound dressing comprising the composite hydrogel of the first aspect of the invention.

A fourth aspect of the invention provides a method of treating a wound comprising the steps of: (a) applying the wound dressing of the third aspect of the invention or the composite hydrogel of the first aspect of the invention to a wound, and (b) displacing the at least one active pharmaceutical ingredient by the method described in the second aspect of the invention.

A fifth aspect of the invention provides a method of delivering an API to a patient comprising the steps of: (a) applying the composite hydrogel of the first aspect of the invention to a patient, and (b) displacing the at least one active pharmaceutical ingredient by the method described in the second aspect of the invention.

### DESCRIPTION

Provided herein is a versatile composite hydrogel that is easy to prepare as it does not require complex, synthetic steps, incorporates low-cost polymers and can work with a wide range of APIs.

As used herein, the term “hydrogel” means a material which is not a readily flowable liquid and not a solid but a gel which is comprised of a gel forming material, such as a hydrophilic polymer that does not dissolve in water. In other words, the hydrogel may be a semi-solid substance. The hydrogel may be formed by the use of a gel forming material, such as a hydrophilic polymer which forms an interconnected, crosslinked network which can entrap, absorb and/or otherwise hold water and thereby create a gel in combination with water.

As used herein, the term “composite hydrogel” means a hydrogel material that physically or covalently incorporates particles, for example active pharmaceutical agents, into a crosslinked network of polymer, wherein the crosslinked network of polymer may comprise one polymer or a combination of one or more different polymers.

The composite hydrogel as described herein comprises an anionic or cationic functional group-containing polymer and at least one active pharmaceutical ingredient (API), wherein the API is oppositely charged to the anionic or cationic functional group-containing polymer. The anionic or cationic functional group-containing polymers may be cross-linked.

The composite hydrogel may further comprise a secondary polymer which may act as a carrier gel matrix or a secondary gel matrix for the anionic or cationic functional group-containing polymer and at least one API. Examples of secondary polymers may include poly-vinyl alcohol (PVA), preferably cryo-crosslinked PVA, poly hexa-methyl methacrylate and agarose.

Preferably the composite hydrogel has a high-water content, such as 20 wt % or more, and/or exists as a gel between the temperatures of about 20° C. and about 40° C., wherein a gel is defined as a semi-solid substance.

As used herein, the term “anionic functional group” means any substituent with an overall negative charge. Without being bound by theory, the present inventors have determined that the anionic functional groups have an overall negative charge when the pKa of the anionic functional groups is lower than the pH of the water or solution (typically about pH 7) in which the anionic functional group-containing polymer is dispersed. When the anionic functional groups of the anionic functional group-containing polymer are negatively charged, they repel each other, so the polymer chains move apart from each other. However, due to cross-linking of the anionic functional group-containing polymers they do not dissolve in the water or solution in which they are dispersed and instead the composite hydrogel swells.

Conversely, when the pKa of the of the anionic functional groups is greater than the pH of the water or solution (typically about pH 7) in which the anionic functional group-containing polymer is dispersed, the anionic functional groups are neutralised and do not have an overall negative charge and therefore the composite hydrogel is not swollen.

As used herein, the term “pKa” refers to the negative base-10 logarithm of the acid dissociation constant (Ka) of a solution i.e., pka=−log₁₀ Ka.

The pKa of a functional group may be measured by measuring the swelling of the hydrogel, wherein when a hydrogel is titrated with an acid, the pka of the functional group is the point at which the hydrogel collapses into a liquid.

Examples of anionic functional groups may include, one or more of hydroxyls, carboxylates, esters, sulfonates, and phosphonates.

Examples of anionic functional group-containing polymers may be include, poly(acrylic acid) (PAA) or poly(acrylic acid) partial sodium salt-graft-poly(ethylene oxide), poly X styrene sulphonic acid, poly X maleic anhydrides or combinations thereof. Other suitable anionic functional group-containing polymers include 2-Acrylamido-2-methylpropane sulfonic acid.

Preferably, the anionic functional group of the anionic functional group-containing polymer comprises at least one carboxylate functional group. More preferably, the anionic functional group-containing polymer is poly(acrylic acid) (PAA).

The anionic functional group-containing polymer may comprise from about 0.5% to about 25% of the anionic functional group by weight of the total anionic functional group-containing polymer, or between 1% and 15%, or preferably between 3% and 8%. The amount of anionic functional group by weight of the total anionic functional group-containing polymer may be measured using common laboratory analytical techniques such as NMR spectroscopy, infrared spectroscopy, x-ray photoelectron spectroscopy, ultraviolet-visible spectroscopy or by titration.

As used herein, the term “cationic functional group” means any substituent with an overall positive charge. Without being bound by theory, the cationic functional groups have an overall positive charge when the pKa of the cationic functional groups is greater than the pH of the water or solution (typically about pH 7) in which the cationic functional group-containing polymer is dispersed. When the cationic functional groups of the cationic functional group-containing polymer are positively charged, they repel each other, so the polymer chains move apart from each other. However, due to cross-linking of the cationic functional group-containing polymers they do not dissolve in the water or solution in which they are dispersed, instead the composite hydrogel swells.

Conversely, when the pKa of the of the cationic functional groups is lower than the pH of the water or solution (typically about pH 7) in which the cationic functional group-containing polymer is dispersed, the cationic functional groups are neutralised and do not have an overall positive charge and therefore the composite hydrogel is not swollen.

The pKa of a functional group may be measured by measuring the swelling of the hydrogel, wherein when a hydrogel is titrated with an acid, the pka of the functional group is the point at which the hydrogel collapses into a liquid.

Examples of cationic functional groups may include, but are not limited to, primary, secondary and tertiary amines, imines and elemental cations such as Ag⁺.

Examples of the cationic functional group-containing polymer may include cationic chitosan, cationic gelatin, cationic dextran, cationic cellulose, cationic cyclodextrin, Polyethyleneimine (PEI), Poly-L-lysine (PLL), Poly(amidoamine) (PAA), Poly(amino-co-ester) (PAE), Poly(2-N,N-dimethylaminoethylmethacrylate) (PDMAEMA) or combinations thereof.

Preferably, the cationic functional group of the cationic functional group-containing polymer comprises at least one amino functional group.

The cationic functional group-containing polymer may comprise from about 0.5% to about 25% of the cationic functional group by weight of the total cationic functional group-containing polymer, or between 1% and 15%, or preferably between 3% and 8%. The amount of cationic functional group by weight of the total cationic functional group-containing polymer may be measured using common laboratory analytical techniques such as NMR spectroscopy, infrared spectroscopy, x-ray photoelectron spectroscopy, ultraviolet-visible spectroscopy or by titration.

As used herein, the term “active pharmaceutical agent” (API) means an ingredient in a pharmaceutical agent which is biologically or pharmacologically active.

The active pharmaceutical ingredient of the composite hydrogel as described herein may be anionic or cationic and is preferably oppositely charged to the anionic or cationic functional group containing polymer. Preferably, the API is a cationic API.

The term cationic API is used to mean an API with an overall positive charge when the pKa of the cationic API is greater than the pH of the solution containing the API.

The term anionic API is used to mean an API with an overall negative charge when the pKa of the anionic API is lower than the pH of the solution containing the API.

The active pharmaceutical ingredient may be an antimicrobial agent. The antimicrobial agent may be an antibacterial agent, an anti-biofilm agent and/or an anti-fungal agent.

Antibacterial agents are molecules that target pathogenic bacteria by well-known methods such as interference with or inhibition of the synthesis of various bacterial cell components or pathways. Examples of suitable cationic antibacterial agents may include any antibiotic comprising functional groups with a pKa greater than the pH of the solution containing the API, for example gentamicin or gentamicin sulfate in water. Examples of suitable anionic antibacterial agents may include any antibiotic comprising anionic functional groups with a pKa less than the pH of the solution containing the anionic antibacterial API. pKa values of common functional groups and APIs are readily available in the literature.

Biofilms are complex structures made up of different bacterial colonies or a single type of cells in a group which proliferate and adhere to a surface. Biofilms are significantly harder to treat than their free-living counter parts and biofilm formation can increase the risk of wound chronicity. Bacteria in the biofilm state are hard to remove by conventional antisepsis, typically requiring 10-1000 times the minimal inhibitory concentration (MIC). Moreover, biofilm bacteria can secrete enzymes such as protease, hyaluronidase and elastase which break down dermal tissue providing a nutrient supply for bacteria and deepening the wound; urease which raises local pH promoting further bacterial growth; and cytotoxins which cause direct cell damage.

Anti-biofilm agents are molecules that prevent the formation of biofilms by well-known methods of action such as targeting bacteria signalling molecules, targeting the extracellular polymeric substance (EPS) surrounding the biofilms, targeting the quorum sensing mechanism between bacteria in the biofilm, cleaving peptidoglycan in the bacterial cell wall, inhibiting biofilm disassembly or cell division, or altering the bacterial membrane.

Examples of suitable cationic anti-biofilm agents may include any anti-biofilm agent comprising cationic functional groups with a pKa greater than the pH of the solution containing the cationic anti-biofilm agents. Examples of suitable anionic anti-biofilm agents may include any anti-biofilm agent comprising functional groups with a pka less than 7.

Anti-fungal agents are molecules that target fungal pathogens by well-known methods such as disruption of the cell membrane, cell division or cell wall synthesis.

Examples of suitable cationic anti-fungal agents may include any anti-fungal agent comprising cationic functional groups with a pka greater than the pH of the solution containing the cationic anti-fungal agents. Examples of suitable anionic anti-fungal agents may include any anti-fungal agent comprising functional groups with a pKa less than 7.

Preferred APIs for use in the composite hydrogel include silver ions, quaternary ammonium cations (QACs), amino glycoside antibiotics, N-acyl homo-serine lactones (AHLs), anti-microbial peptides (AMPs), Polymyxin antibiotics, enzymes, Polyhexanide (also known as polyhexamethylene biguanide, PHMB), beta-lactam antibiotics and/or combinations thereof.

The quaternary ammonium cation (QAC) may be cetrimide, and/or the amino glycoside antibiotic may be gentamicin, such as gentamicin sulfate, or streptomycin and/or the Polymyxin antibiotic may be polymyxin B and/or the beta-lactam antibiotic may be amoxicillin. The antimicrobial peptide (AMP) may be a defensin, for example, the antimicrobial peptide may be beta-defensin.

Other suitable APIs may include anti-cancer agents, anti-inflammatory agents, tissue regenerative APIs, gene therapies, antibodies and/or combinations thereof.

The API may be present in an amount at least as high as the minimal inhibitory concentration (MIC) of the microbe (e.g., bacteria) or the minimal effective concentration (MEC) of the API or the minimal bactericidal concentration (MBC) of the API

The minimum inhibitory concentration (MIC) of the microbe (e.g., bacteria) is the lowest concentration of a substance which prevents the visible growth of a microbe (e.g., bacteria). MIC is often measured in micrograms per millilitre (μg/mL) or milligrams per litre (mg/L).

The minimal effective concentration (MEC) is the minimum plasma concentration of an API needed to achieve a sufficient API concentration at receptors to produce the desired pharmacologic response. MEC is often measured in milligrams per millilitre (mg/mL) or milligrams per litre (mg/L). The MEC may be measured in wound exudate or wound tissue plasma.

The minimal bactericidal concentration (MBC) is the lowest concentration of an antibacterial agent required to kill a bacterium. MBC is often measured in colony-forming units per millilitre (CFU/mL).

The anionic or cationic functional group-containing polymer and the at least one API can be bound together to form an anionic or cationic functional group-containing polymer-API complex.

The anionic or cationic functional group-containing polymer-API complex may be bound by non-covalent interactions such as Coulombic interaction, hydrogen bonds, hydrophobic interactions, ionic bonds, dipole-dipole bonds. Preferably, the anionic or cationic functional group-containing polymer-API complex is bound by Coulombic interactions.

As used herein the term “Coulombic interaction” means the electrostatic interaction between electric charges.

For example, there may be a Coulombic interaction between an anionic functional group of the polymer and a cationic API at a given pH and/or a Coulombic interaction between a cationic functional group of the polymer and an anionic API at a given pH.

When the functional group of the polymer is anionic, the pKa of the anionic functional group is lower than the pKa of the cationic API.

When the functional group of the polymer is cationic, the pKa of the cationic functional group is higher than the pKa of the anionic API.

Without being bound by theory, it is believed that when the API comprises cationic functional groups, the greater the number of cationic groups i.e., functional groups with a pKa greater than 7, then the greater the binding energy of the API to the composite hydrogel matrix. Equally, when the API comprises anionic functional groups, the greater the number of anionic groups i.e., functional groups with a pKa less than 7, then the greater the binding energy of the API to the composite hydrogel matrix.

The anionic or cationic functional group-containing polymer-API complex is dispersed in the composite hydrogel.

The composite hydrogel described herein may comprise any natural or synthetic polymer suitable for use in a composite hydrogel.

Examples of suitable natural polymers include hyaluronic acid, chitosan, heparin, alginate, fibrin and/or combinations thereof.

Examples of suitable synthetic polymers include polyvinyl alcohol, polyethylene glycol, sodium polyacrylate, acrylate polymers and/or copolymers thereof.

The polymer may be crosslinked.

The composite hydrogel can be formed by mixing the natural or synthetic polymer with water in an amount of at least 10%, at least 15%, at least 20%, at least 25% or at least 30% by weight of the composite hydrogel and the at least one API.

The composite hydrogel may further comprise a diagnostic agent. Any appropriate diagnostic agent that produces a visible signal in response to an infection may be used. For example, the diagnostic agent may be a dye or a pH indicator or otherwise visualisable diagnostic agent that can only be visualised when an infection is present. The visualisable element may be as simple as a colour change in response to the presence of an infection, such dyes are well known. Or it might be for example a fluorescent signal that requires illumination with UV light to be visible, such a fluorescent dye.

Other possible diagnostic agents include organic or inorganic dyes such as pH sensitive dyes such as crystal blue, bromothymol blue, methyl orange, calcein and other pH or concentration sensitive fluorometric dyes.

Alternatively, other chemical diagnostic agents, such as redox signalling molecules may be included.

The API may be displaced from the composite hydrogel by a method which comprises the step of treating the composite hydrogel with an ion source comprising anions, cations, or a combination thereof. Preferably, the ion source comprises protons.

As used herein, the term “ion source” means a source which delivers ions and/or molecules that readily dissociate in aqueous media to provide ions.

Examples of suitable ion sources may include cold atmospheric plasma (CAP), plasma activated water, plasma or dielectric barrier discharge, glow discharge, an acid and/or a mineral solution. Suitable acids include but are not limited to organic acids or mineral acids such as nitric acid hydrochloric acid, or citric acid. Suitable mineral solutions include but are not limited to calcium chloride, magnesium chloride or aluminium chloride. Preferably, the ion source is cold atmospheric plasma.

As described above, functional groups of like charge of the functional group-containing polymers repel each other, so the polymer chains move apart from each other. However, due to cross-linking of the polymer chains, they do not dissolve in the water or solution in which they are dispersed, instead the composite hydrogel swells. Conversely, when the functional groups are not charged the composite hydrogel is not swollen.

Without being bound by theory it is believed that treatment of the composite hydrogel with an ion source comprising anions and/or cations increases the osmotic concentration of the carrier gel. This is because an increase in ions outside the composite hydrogel leads to an increase in ions inside the composite hydrogel therefore reducing the osmotic gradient. These ions may screen repulsive charges between the functional groups of like charge of the functional group-containing polymers. This charge screening leads to a de-swelling or collapse of the composite hydrogel and displacement of the API from the composite hydrogel. It is believed that this effect is stronger with divalent or trivalent cations compared to monovalent cations.

The term osmotic concentration or osmolarity is a measure of solute concentration. Osmotic concentration is typically measured in osmoles of solute per litre of solution (Osm/L).

In addition, protonation by protons in the ion source may disrupt the Coulombic interaction between an anionic species and a cationic species because the electrostatic attraction between the two charged species is lost. Accordingly, the coulombic attraction of the anionic functional-group containing polymer with the oppositely charged API, or the coulombic attraction of the anionic API with the opposite charged functional group-containing polymer is disrupted. Therefore, the API may be displaced from the composite hydrogel.

Cold Atmospheric Plasma (CAP) is a nonthermal plasma, a type of non-equilibrium plasma in which the gas remains at relatively low temperature relative to the temperatures that are generated in thermal plasmas. Non-thermal plasmas are weakly ionised plasmas. CAP can be sustained in an inert gas for example argon, helium, nitrogen or air. When CAP is sustained in air, or when an inert gas CAP is launched into air, the result is a mix of oxygen, nitrogen and hydrogen radicals, ions, electrons, photons and ultraviolet (UV) radiation.

As used herein, the term “plasma” means plasma operated at around atmospheric pressure with the temperature of the plasma gas typically less than about 60° C., preferably less than about 45° C., and ideally less than about 37° C.

The plasma can be formed using any plasma apparatus that generates a plasma stream that can be directed at a surface to be treated. The plasma apparatus may form a plasma jet, torch, needle or a dielectric barrier discharges (DBDs) such as a floating electrode configuration for treating a surface. Atmospheric pressure plasma jet devices are known in the art. Plasma jet devices can be fabricated in a multitude of electrode configurations and can be operated over a wide range of power and frequency (Hz to GHz) settings. For example, the plasma jet can be operated at a frequency of from about 10 kHz to about 30 MHz, a voltage of from about 5 kV to about 15 kV and a gas flow rate of about 0.5 litres per minute to about 10 litres per minute. The skilled person will easily be able to select suitable CAP parameters for a particular application.

The cold atmospheric plasma is excited and sustained by the application of radio frequency or microwave electrical power.

As used herein the term excited refers to when atoms and molecules in a gas become ionised, so that positive ions and free electrons result.

As used herein the term sustained refers to a continual application of radio frequency or microwave electrical power such that particles are continually ionised and the plasma is maintained.

A typical plasma jet device comprises two or more coaxially placed electrodes defining a plasma chamber there between. A plasma jet can be generated at an open end of the device by introducing a flow of gas at the other end of the device while a sufficient voltage is applied between the electrodes. A nozzle can be used at the open end to converge the plasma jet in order to obtain higher plasma densities. The plasma apparatus further comprises a power supply device for supplying electric power to the electrodes to produce plasma in the plasma chamber.

The plasma may be formed from an inert gas such as helium, argon or molecular gases such as oxygen, nitrogen, air or a combination of an inert gas with air, oxygen and/or water vapour. Optionally, the gas may also comprise an additive, such as an additive for improving the wound healing, improving the plasma characteristics or providing a sterilising effect.

As used herein, the term “inert” means a gas that does not undergo chemical reaction under given conditions.

The gas flow into the plasma chamber of the plasma apparatus is preferably controlled by a flow controller and/or an inlet valve which is arranged between a gas source and the gas inlet of the plasma apparatus.

Alternatively, the plasma can be operated in ambient air with no mechanical and/or physical control over the gas flow.

Optionally, the plasma apparatus has an ability to modulate an output to the electrodes. With this output modulation, it is possible to change the state of plasma. Note here that the output modulation refers to altering the output in characteristics to thereby change the plasma state such as pulsating the output, increasing or decreasing the magnitude of output, turning on and off the output, changing output frequency or like processing.

The plasma has a gas temperature typically below about 60° C., or preferably below about 45° C. and below about 37° C. when measured on the treated surface.

Without being bound by theory, it is believed that the CAP argon jet creates reactive oxygen species, primarily hydrogen peroxide which pass into the composite hydrogel matrix as a flux, but in addition there is evidence that the CAP creates oxygen and nitrogen species, for example nitric acid, nitrous acid and peroxynitric acid via interaction of the plasma with atmospheric nitrogen.

As described above, it is believed that the ion source provided by the CAP argon jet increases the osmotic concentration of the carrier gel, leading to charge screening between the functional group-containing polymers and de-swelling or collapse of the composite hydrogel. Consequently, the API is displaced/pumped out from the composite hydrogel.

Additionally, the ion source may lead to a local decrease in pH from about pH 7 to about pH 5 or less, or preferably about pH 4.5 or less which may protonate the anionic functional groups on the anionic functional-group containing polymer or the anionic API. The protonation may disrupt the coulombic attraction of functional-group containing polymer with the oppositely charged API, and reduces the interchain repulsion of the polymer chain. This decrease in repulsion creates a physical collapse of the composite hydrogel and displacement of the API. The flux of the excited plasma gas helps to carry the released API out of the composite hydrogel and into the surrounding environment, which may be water, or surrounding wound tissue.

The ion source may be applied to the composite hydrogel for a period of from about 1 minute to about 10 minutes, or from about 3 minutes to about 8 minutes, or for about 5 minutes.

Also provided herein is a wound dressing comprising a composite hydrogel comprising an anionic or cationic functional group-containing polymer and at least one API, wherein the API is oppositely charged to the anionic or cationic functional group-containing polymer.

Suitable types of wound dressing may include bandages, woven or non-woven gauze or sponge. The wound dressing may be coated or impregnated with the composite hydrogel. The wound dressing may comprise a single layer or multiple layers of the composite hydrogel. Alternatively, the composite hydrogel may form the entirety of the wound dressing.

The wound dressing or the composite hydrogel may be used in treating a wound. As used herein, the term “wound” refers to all types of tissue injury, including those resulting from chronic medical conditions. The wound may be an open wound and may be acute or chronic. The wound may be on the inside or on the outside of the body. Wound dressings as described herein can be used for the treatment of acute wounds, chronic wounds, surgical wounds, ulcers, ulcerating cancers, thermal wounds, chemical, radiation-induced wounds, and superficial burns.

The composite hydrogel may be applied directly to a wound or it may be applied to a wound dressing which is then applied to a wound.

The composite hydrogel may also be applied to a nail, such as a fingernail or toenail, for example to treat fungal nail infections such as onychomycosis. The composite hydrogel may be dissolved in a suitable solvent and painted onto a nail surface before being treated with CAP to release the API. Suitable solvents may include one or more of acetone, ketones, aldehydes, organic acids (such as carboxylic acid) and/or alcohols and suitable APIs for treating fungal nail infection may include allylamine. The composite hydrogel may also be applied directly onto a nail surface or applied directly onto the nail surface using a suitable adhesive before being treated with CAP to release the API.

Wherein the composite hydrogel includes an API comprising antibodies or a gene therapy, the oppositely charged functional group-containing polymer may be a dendrimer such as poly(amidoamine) or part of an advanced drug delivery system. The dendrimer or advanced drug delivery system may act as a carrier or drug delivery vehicle to deliver the API to the API target site which may be on the inside or on the outside of the body.

In this scenario, the dendrimer may be bound by electrostatic charges within an anionic polymer (with a hydrogel carrier) as previously described. On application of the ion/proton source, the anionic polymer collapses releasing both dendrimer and the API, such as a gene therapy or antibody.

Provided herein is a method of treating a wound comprising the steps of:
(a) applying the wound dressing or the composite hydrogel comprising an anionic or cationic functional group-containing polymer and at least one oppositely charged API to a wound, and
(b) displacing the at least one oppositely charged API by the method as described herein, which comprises the step of treating the composite hydrogel with an ion source.

The wound dressing or the composite hydrogel can be applied over a wound or on a region of skin to be treated therapeutically. The wound dressing or the composite hydrogel is then treated with an ion source, preferably cold atmospheric plasma.

Accordingly, the plasma apparatus is preferably configured so that the non-thermal plasma emitted therefrom contacts the surface of the wound dressing and/or the composite hydrogel and the plasma that passes through the wound dressing and/or the composite hydrogel displaces the API from the composite hydrogel to deliver it to the wounds or region of skin to be treated and improve the wound healing.

Provided herein is a method of delivering an API to a patient comprising the steps of:
(a) applying the composite hydrogel comprising an anionic or cationic functional group-containing polymer and at least one oppositely charged API to a patient, and
(b) displacing the at least one API by the method as described herein, which comprises the step of treating the composite hydrogel with an ion source.

Typically, the composite hydrogel is applied topically to a patient's wound or on a region of skin to be treated therapeutically. The composite hydrogel can then treated with an ion source, preferably cold atmospheric plasma.

### BRIEF DESCRIPTION OF THE DRAWINGS

The invention will now be described in detail, by way of example only, with reference to the figures, in which:

FIG. 1 shows the results of quantification of gentamicin release from the composite hydrogel following 2 minutes plasma jet treatment. Error bars represent standard deviation (n=3) plotted in Origin. Students t-test was carried out to assess statistical significant p<0.001.

FIG. 2 shows viable cell count of 8 h P. aeruginosa (PAO1) biofilms after 18 h incubation with composite hydrogels loaded with water with and without CAP activation and gentamicin loaded composite hydrogels (A) cetrimide loaded gels (B) and silver nitrate loaded gels (C) with and without CAP activation relative to untreated control biofilms. Error bars show standard deviation (n=3).

FIG. 3 shows viable cell count of 8 h S. aureus (H560) biofilms after 18 h incubation with composite hydrogels loaded with water with and without CAP activation and gentamicin loaded composite hydrogels (A) cetrimide loaded gels (B) and silver nitrate loaded gels (C) with and without CAP activation relative to untreated control biofilms. Error bars show standard deviation (n=3).

FIG. 4 shows zone of bacterial growth inhibition (ZOI) of Gentamicin loaded composite hydrogels with and without CAP activation compared to H₂O loaded composite gels with CAP activation. P. aeruginosa (PAO1) (A), S. aureus (H560) (B) and E. faecalis (JH2-2) (C). Error bars represent standard deviation (N=3) and a One-way ANOVA was carried out using GraphPad 8.0. (****) p<0.0001.

FIG. 5 shows ZOI of Silver Nitrate loaded composite hydrogels with and without CAP activation compared to H₂O loaded composite gels with CAP activation. P. aeruginosa (PAO1) (A), S. aureus (H560) (B) and E. faecalis (JH2-2) (C). Error bars represent standard deviation (N=3) and a One-way ANOVA was carried out using GraphPad 8.0. (**) p<0.01.

FIG. 6 shows ZOI of Polymyxin B loaded composite hydrogels with and without CAP activation compared to H₂O loaded composite gels with CAP activation. P. aeruginosa (PAO1) (A), S. aureus (H560) (B) and E. faecalis (JH2-2) (C). Error bars represent standard deviation (N=3) and a One-way ANOVA was carried out using GraphPad 8.0. (**) p<0.01.

FIG. 7 shows ZOI of Cetrimide loaded composite hydrogels with and without CAP activation compared to H₂O loaded composite gels with CAP activation. P. aeruginosa (PAO1) (A), S. aureus (H560) (B) and E. faecalis (JH2-2) (C). Error bars represent standard deviation (N=3) and a One-way ANOVA was carried out using GraphPad 8.0. (*) p<0.1.

FIG. 8 shows a schematic illustration of multiple potential coulombic interactions between carboxylate groups on the PAA with protonated primary amines on the peptide chain of polymyxin B. Although only the primary amines are shown as protonated for clarity, many of the secondary amines are likely to be also protonated and have significant coulombic interaction with the PAA.

FIG. 9 shows a proposed mechanism of release of antimicrobial (in this illustration gentamicin) via effect of the CAP protonating carboxylate groups, releasing the bound API and the increased external osmotic pressure on the PAA particles causing de-swelling and ‘pumping’ out of the API.

FIG. 10 shows SEM image of silver loaded PAA particle (A). EDX of silver loaded PAA particle: colours are: Yellow (Ag); Pink (AI); Blue (Na); Green (O); Red (C).

FIG. 11 shows a schematic of Fluorogent synthesis.

FIG. 12 shows a LSCM image of PAA particle loaded with Fluorogent prior to sectioning (A). Sectioned PAA particle. Note largely even distribution of fluorescence through particle in contrast to silver (B).

FIG. 13 shows a schematic of procedure for preparation of antimicrobial loaded PAA/PVA composite gels.

FIG. 14 shows a schematic of the plasma jet set up.

FIG. 15 shows an AMPS (2-Acrylamido-2-methylpropane sulfonic acid) crosslinked hydrogel matrix with ionic interactions of Na⁺ and M⁺ (cationic model drug) with sulfonic acid group in media.

FIG. 16 shows cold plasma exposure on the AMPS containing hydrogel and subsequent drug release.

FIG. 17 shows (A) gentamicin cumulative release (ug/ml) from plasma exposed AMPS containing hydrogel against control hydrogel for 6 hours and (B) total gentamicin release from plasma exposed AMPS containing hydrogel against control hydrogel in 6 hours.

### EXAMPLES

To demonstrate the system versatility the release of five different antimicrobial moieties is demonstrated, with release (where possible) being quantified both via colorimetric assay and microbiology to demonstrate efficacy against bacterial species Staphylococcus aureus (S. aureus) and Pseudomonas aeruginosa (P. aeruginosa) which are commonly isolated from chronic wounds.

### Example 1

### Materials and Methods

### Materials

Sodium polyacrylate (PAA), polyvinyl alcohol (PVA), gentamicin sulphate salt and streptomycin sulphate were all purchased from Sigma (Poole, UK). Cetrimide was donated from Novo Nordisk Pharmatech. Tryptic soy agar (TSA), Luria-Bertani (LB) agar, Muller-Hinton (MH) agar, brain-heart infused (BHI) agar, tryptic soy broth (TSB), LB broth, MH broth were all purchased from Sigma. Whatman Polycarbonate membranes were purchased from Fischer.

### PAA Polymer

‘Superabsorbent’ polyacrylic acid partial sodium salt-graft-poly(ethylene oxide) particles (CAS number: 9003-04-7) was obtained from Sigma-Aldrich. A further source of PAA was obtained from BASF, with the tradename Saviva™. Saviva particles are chemically similar to the Sigma-Aldrich particles (primarily polyacrylic acid) but with a smaller particle size distribution and more spherical morphology.

### Bacterial Strains and Growth Conditions

Pseudomonas aeruginosa (P. aeruginosa PAO1) Staphylococcus aureus (S. aureus H560) from the Jenkins collection at the University of Bath. Enterococcus faecalis (E. faecalis JH2-2) from the Gebhard collection at the University of Bath. Bacteria were maintained in 15% (v/v) glycerol at −80° C. and streaked out as required onto Luria-Bertani (LB) agar, tryptic soy agar (TSA) and brain-heart infused (BHI) agar respectively. Overnight cultures were made by inoculating a single bacterial colony into 10 mL of broth, Muller-Hinton (MH) broth for Kirby-Bauer tests and MIC assays or LB broth, tryptic soy broth (TSB) and BHI broth respectively for biofilm work.

Loading of PAA Particles with Antimicrobials

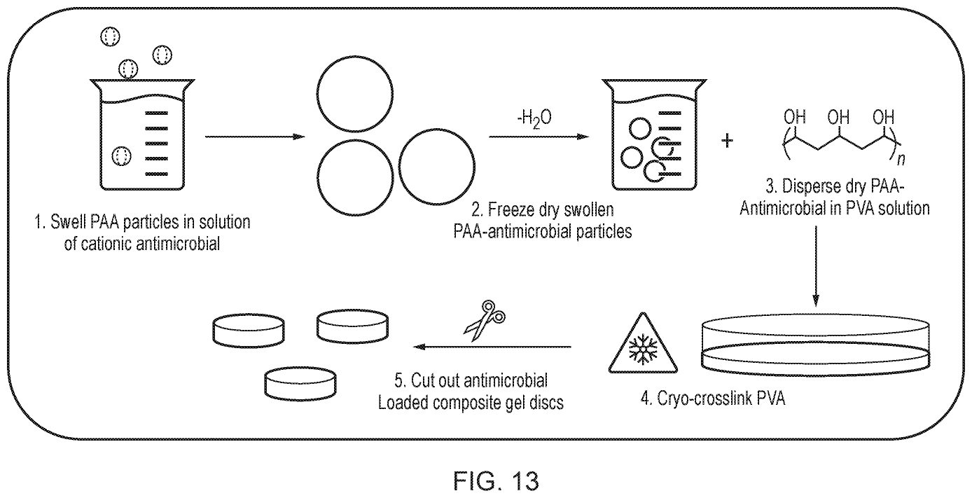

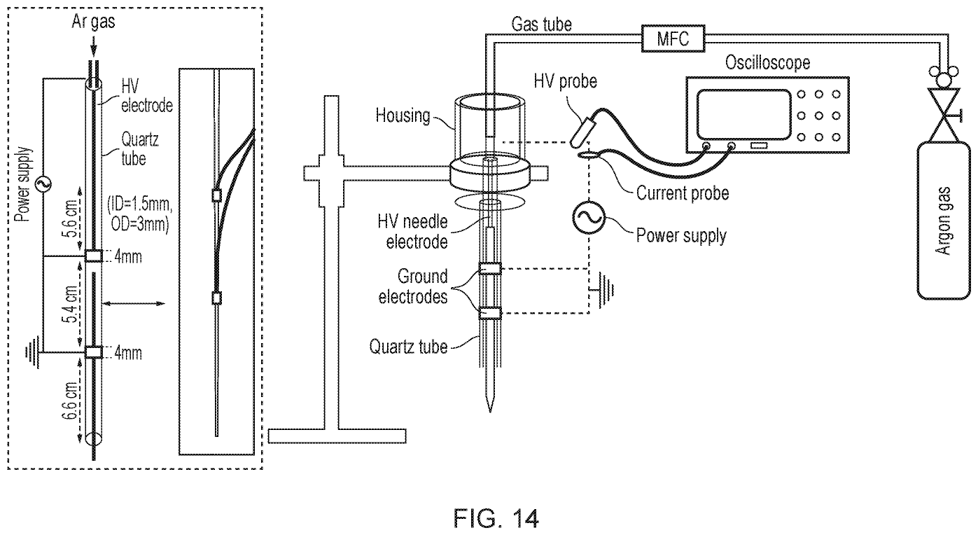

The film loading and preparation process is shown schematically in FIG. 13. Solutions of silver nitrate, streptomycin, gentamicin sulfate or polymyxin B were made up in sterile MilliQ water to final concentration of 1 mg/mL, Cetrimide was dissolved in MilliQ water at 10% (and 1% w/v) and Silver Nitrate was made up to 0.01 M in MilliQ water. 1 g of PAA was added to 100 ml of the desired antimicrobial 1% (w/v) in a round bottom flask. The flask was left for ca. 1 hour to allow for complete swelling of the PAA particles. 20 mL then heated under vacuum in a rotary evaporator to around 95° C. to boil off most of the water. The remaining wet gel was then frozen in liquid nitrogen before being thawed under vacuum (ca. 50 bar). This freeze/thaw cycle was repeated three times. Finally, around 10 ml of ethanol was added to the PAA to form a water-ethanol azeotrope and was heated to 60° C. and this was taken through a two further freeze/thaw cycles under vacuum to yield a dry product. The product was then crushed in a pestle and mortar to a coarse powder.

### Preparation of PAA/PVA Composite Hydrogel

0.1 g of the drug loaded PAA powder was mixed with dry PVA (5% (w/v)) to obtain a homogenous powder and then is dissolved at 95° C. for 1 h. 20 ml of the PAANaPA/PVA solution was then added to a 20.5 cm diameter petri dish and spread evenly. The gels were placed into a −20° C. freezer until frozen and removed and defrosted at 25° C. this process was repeated twice more to enable cryo-crosslinking of the PVA.

### Ar-CAP Jet Set Up and Operating Parameters

The Ar-CAP jet shown in FIG. 14 consisted of an internal steel needle electrode (outer diameter=0.9 mm, inner diameter=0.6 mm, length 51 mm) sealed inside a quartz tube (inner diameter=1.5 mm, outer diameter=3 mm). Two external copper electrodes of length 4 mm 5.6 cm from electrode, spaced 5.4 cm apart and 6.6 cm from the bottom of the tube. Ar gas was kept at 1.0 standard litres per minute (SLPM) and generated at 10 kV at 23.5 kHz. Voltage and current waveforms were monitored using oscilloscope. The gap distance was 1.5 cm and the Ar-CAP jet was stationary unless otherwise stated. 31

### Kirby-Bauer Test

Bacterial overnight was made as outlined, subcultures were made in fresh MH broth absorbance corrected to OD 0.5 (˜1×10⁷1×10⁷ CFU/mL). 100 μL was added to Muller-Hinton agar to and spread to create a bacterial lawn. Gel discs were placed in the centre of the plate and either treated with Ar-CAP or left untreated. Plates were incubated at 37° C. for 18 h, facing up to prevent the gel from moving. Zone of inhibition (ZOI) was measured and corrected to the diameter of the gel.

### Ninhydrin Assay for Aminoglycoside Release

Aminoglycoside was made up in pH 7.4 assay buffer (39.1 mL 0.2 M sodium hydroxide solution was added to 50 mL 0.2 M monobasic potassium phosphate solution and made up to a final volume of 200 mL with DI H₂O) to starting concentration of 1 mg/mL. A concentration range was then made (1000 μg/mL-100 μg/mL). 500 μL of aminoglycoside was added to 500 μL of 1% (w/v) ninhydrin solution. This was then incubated at 95° C. for 45 minutes. 200 μL was aliquoted into a round bottom 96-well plate and the absorbance was read at 560 nm (Clariostar, Omega). Values were blank corrected to 200 500 μL of pH 7.4 buffer with 500 μL 1% (w/v) ninhydrin solution. This was repeated in triplicate.

### Treatment of Early-Stage Biofilms

Bacterial overnights (ON) were grown as before in LB, TSB and BHI agar. ON were spun down at 4000 rpm for 10 minutes and resuspended in 10 mL sterile PBS (pH 7.4, 25° C.). 10 μL of ON was added to 10 ml of sterile PBS to as a starting OD of 0.1 (˜ 1×10⁵ CFU/mL). Whatman polycarbonate membranes (pore size 0.2 diameter 19 mm) were placed onto BHI agar plates, shiny side up and sterilised for 10 mins using UV-C. 20 μL of artificial wound fluid was added to the membrane. Then 30 μL of bacterial subculture was added. The membranes were subsequently incubated at 37° C. for 8 h to grow an early-stage biofilm.

### SEM/EDX Imaging and Laser Scanning Confocal Microscopy

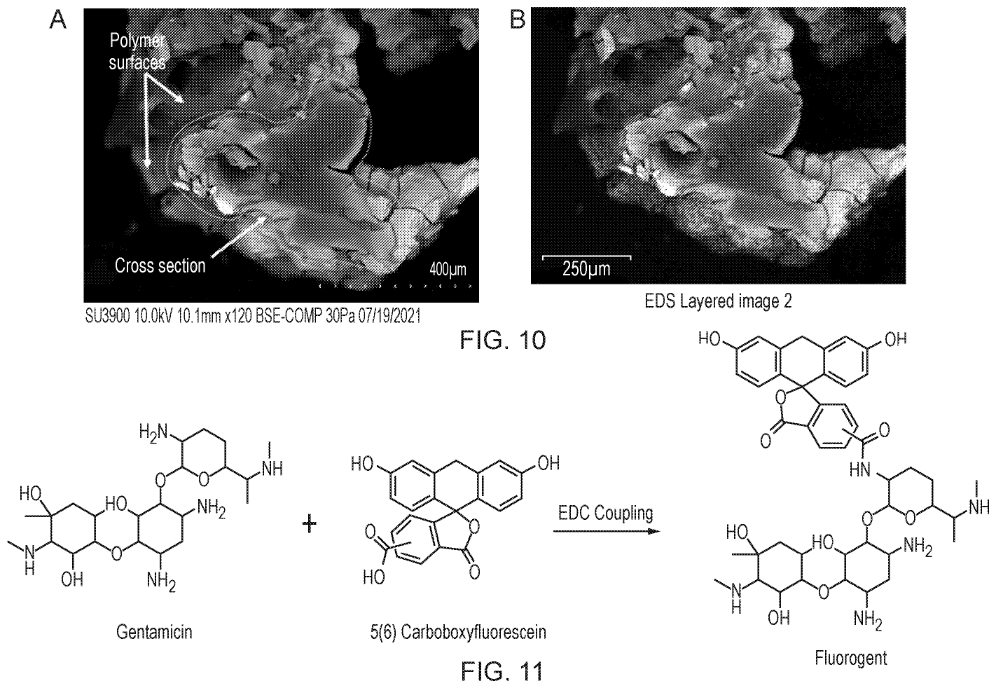

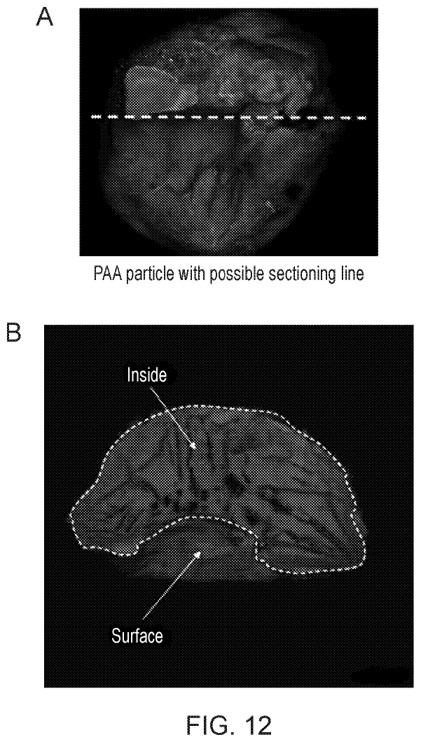

PAA particles were loaded with silver nitrate, cut in half and mounted on a stainless steel stud using carbon tape without any conductive coating. For imaging and compositional analysis, Hitachi SU3900 large chamber, variable pressure SEM, equipped with energy dispersive X-ray analyser (Oxford Instruments AzTec 170 mm²) was used (FIG. 10). For LSCM analysis, PAA particles loaded with fluorescently labelled Gentamicin (both entire particle and half-cut particle for the surface and cross-sectional analysis respectively) were prepared and analysed using LSM800 confocal microscope. The microscope was equipped with Airyscan and Multiphoton laser with the excitation cut-off wavelength of 405 nm (FIG. 12).

### Results and Discussion

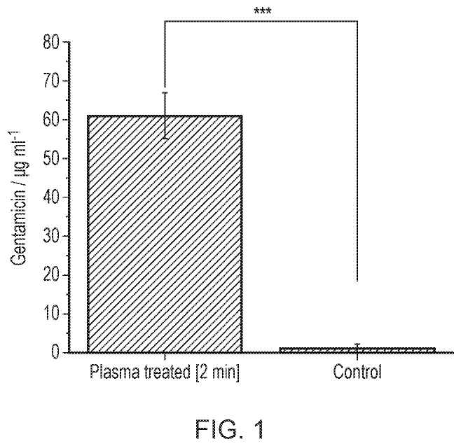

Gentamicin is an aminoglycoside antibiotic used both systemically and topically. Its relatively high systemic toxicity means it is often use preferentially for external application, for example to treat Otitis media and skin/wound and diabetic foot infections. Gentamicin contains a net positive charge at pH 7, due to protonation of its two secondary amine groups (pka=8.8 and 9.9). Ninhydrin was used to quantify release of gentamicin both prior to CAP treatment (passive release) and following CAP treatment. Ninhydrin reacts with primary and secondary amines to form Ruhemann's purple, a coloured precipitate which is detected spectrophotometrically at 540 nm. The Ninhydrin assay was used to quantify release of gentamicin with (triggered release) and without CAP treatment (passive release). FIG. 1 shows the passive and triggered release of gentamicin from the composite gel. After two minutes treatment with Ar CAP jet, 61 mg ml⁻¹ (+/−10 mg ml⁻¹) gentamicin was released from gel into the surrounding PBS whereas only 1.1 mg/mL was passively released. (Gentamicin concentration was calculated from standard curve in ESI) The Minimal Inhibitory Concentration (MIC) of gentamicin against most clinically important bacteria is in the range of 2-4 mg ml⁻¹ for many clinical strains of S. aureus and P. aeruginosa.

Effects on Early-Stage P. aeruginosa & S. aureus Biofilms

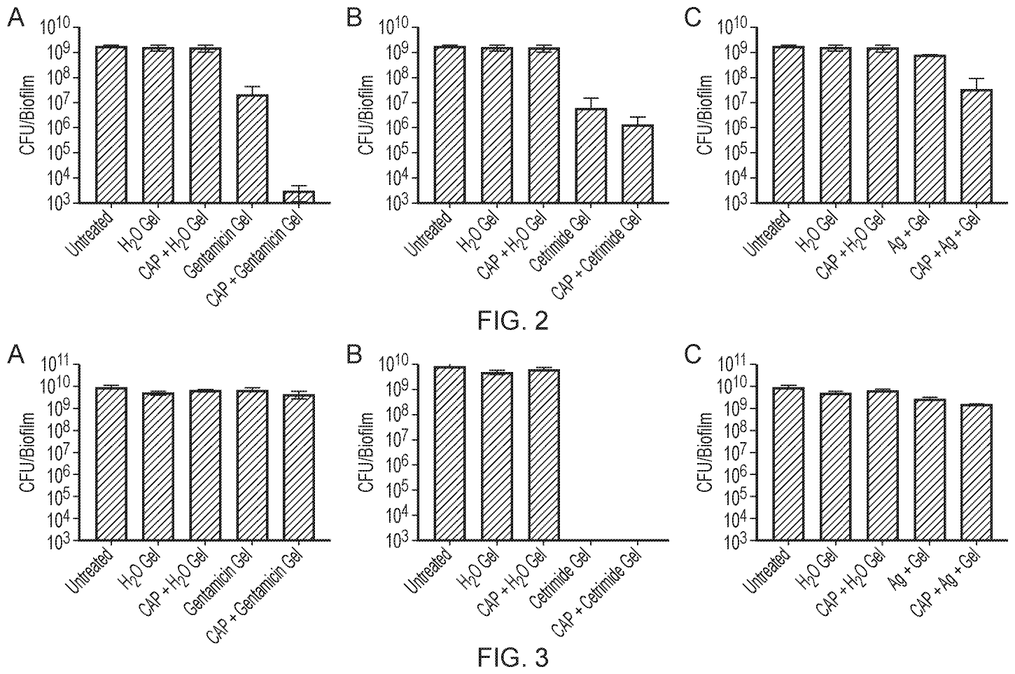

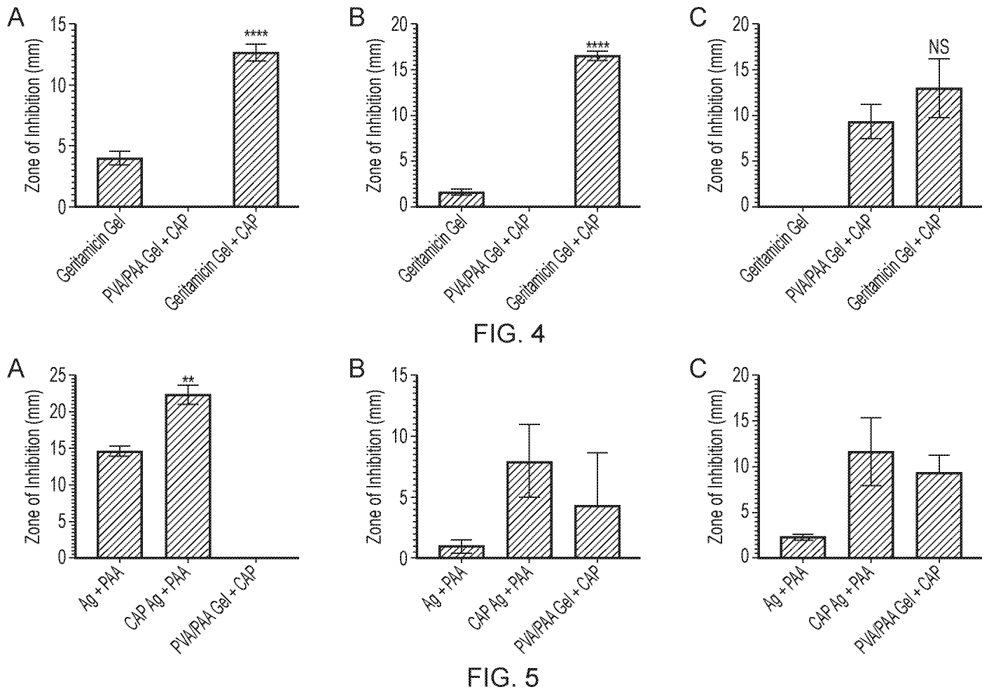

To better test the composite gel system in clinically relevant conditions, early-stage P. aeruginosa biofilms as per protocol. FIG. 2 shows a 2-log reduction in viable cells for the gentamicin gel without application of the plasma jet, however greater than a 5-log reduction in vi able cells when treated with the CAP activated composite gentamicin loaded gel. To ensure that the results observed were as a result of CAP stimulated gentamicin release and not simply from the CAP, a composite gel loaded with water only was applied to the biofilms with and without CAP activation. No reduction was observed in the water loaded compositive gel with or without CAP activation.

The gentamicin and silver nitrate gels were ineffective on S. aureus early-stage biofilms (FIG. 3). Interestingly, the cetrimide gel completely eradicated the S. aureus biofilms.

Subsequent experiments were carried out to determine relative release of a range of cationic antimicrobials by measuring their antimicrobial efficacy against three key bacterial species and strains: S. aureus (H560), P. aeruginosa (PAO1) and Enterococcus faecalis (E. faecalis) (JH2-2).

### The Kirby-Bauer Test

The Kirby-Bauer (KB) test is a standard test used to assess the susceptibility of bacteria to different antimicrobials by measuring the zone of bacterial growth inhibition (ZOI) due to the out diffusion of the antimicrobial agent from a sterile disc soaked in that compound. In this case, a modified form of the KB test was performed. Plasma activate antimicrobial loaded, composite hydrogels were compared to non-activated antimicrobial loaded, composite hydrogels and non-loaded, composite hydrogels with and without plasma activation. The initial system studied was a gel containing 1 mg ml⁻¹ gentamicin (Error! Reference source not found.). The measured ZOI showed that the gentamicin gel without plasma activation resulted in a 4 mm ZOI, which is thought to be because of passive gentamicin release from the hydrogel. The non-loaded hydrogel with plasma activation has a ZOI of 0 mm confirming that the without plasma activation showed no measurable ZOI.

### Loading and Release of Other Cationic Antimicrobials: Silver, Cetrimide and Polymyxin B

One of the unique features of this composite gel delivery system is that the architecture allows for a range of drugs to be encapsulated and delivered. While further work seeks to further optimize the loading parameters the results below show successful encapsulation and delivery of silver ions, Polymyxin B and Cetrimide.

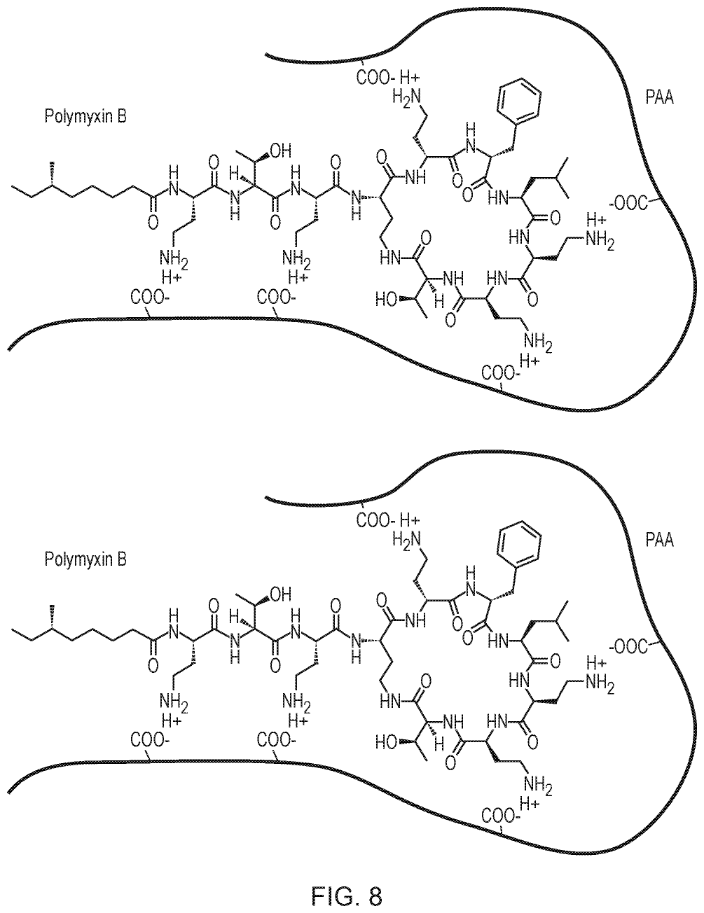

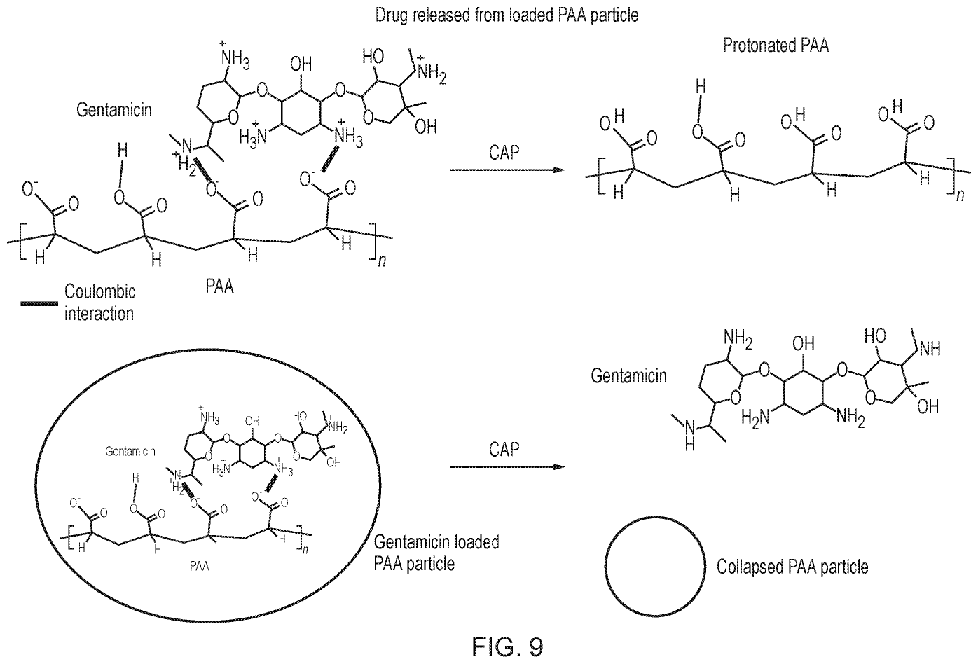

Results show that the principle of drug encapsulation with the PAA matrix and subsequent CAP mediated release appears to be effective. The variation in bacterial susceptibility to the plasma jet applied to the unloaded gel is interesting and is thought to related to the varying susceptibility of different species to the RONS generated by the CAP. Sensitivity to different antimicrobials varied as a function of both the antimicrobial moiety and the bacterial species. In this case, P. aeruginosa is seen to be sensitive to Ag⁺ passively leaching from the composite gel, while S. aureus and E. faecalis are not. E. faecalis appears to be less sensitive than P. aeruginosa and S. aureus to Cetrimide. Interestingly, Polymyxin B seemingly has relatively no passive release when compared to the antimicrobials, this is potentially as a result of stronger (additive) interaction with the PAA, as it has multiple cationic groups, vide infra (FIG. 8).

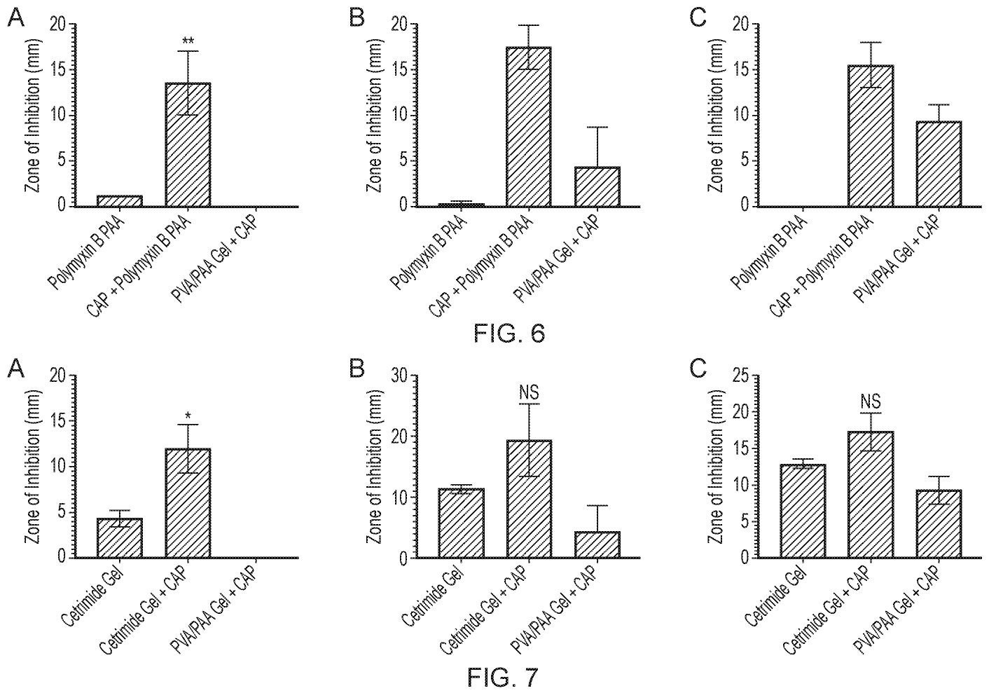

Relative passive leaching of different antimicrobials can be qualitatively assessed by looking at FIG. 5-7. The difficulty in cross comparison of the different antimicrobial susceptibility of the bacterial species to the various antimicrobials tested. The key comparison here is to look at the ratio of ZOI of the antimicrobial loaded composite gel (1^{st} column in graphs below) to the zone of inhibition for the same loaded gel following application of the CAP jet 2^{nd} column). The increase in zone of inhibition following CAP application is tabulated in Table 1.

TABLE 1
Ratio of bacterial zone of inhibition following application of plasma
jet to antimicrobial loaded gel over non-plasma activated g

| | P. aeruginosa | S. aureus | E. faecalis |
|---|---|---|---|
| Gentamicin | 3.6 | 8.2 | >10 |
| Silver | 1.5 | 7 | 6 |
| Cetrimide | 2.7 | 1.8 | 1.3 |
| Polymyxin B | 13 | 34 | >30 |

The ratios shown in Table 1 show that Polymyxin B, followed by gentamicin appear to be strongly bound with PAA matrix with little passive leaching. Ag⁺ Silver and Cetrimide appear to show a much greater degree of passive out-diffusion from the gel.

### Example 2-Plasma Induced Release of Gentamicin from a AMPS Containing Hydrogel (2-Acrylamido-2-Methylpropane Sulfonic Acid)

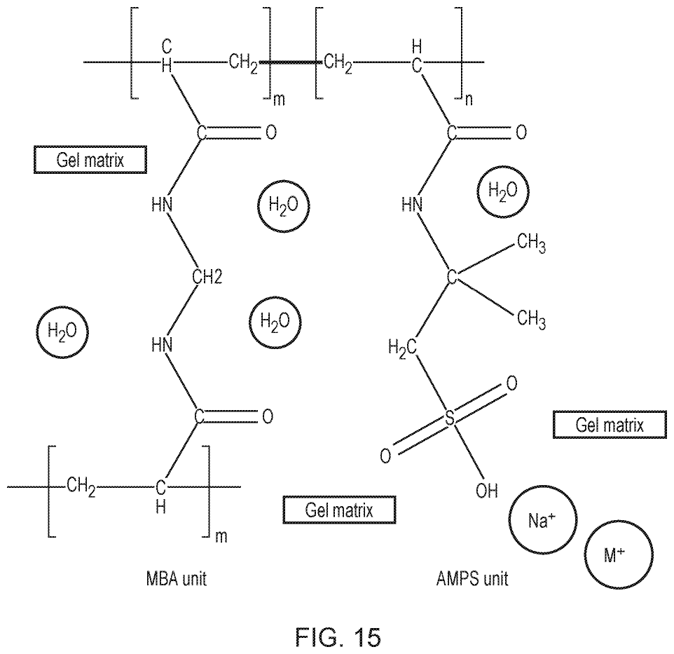

An AMPS (2-Acrylamido-2-methylpropane sulfonic acid) crosslinked hydrogel matrix (see FIG. 15) was prepared by first dissolving AMPS in water at room temperature and then adding NaOH to neutralise the sulfonic acid to form Na-AMPS in situ. Then chain initiator potassium persulfate was added to generate radicals, which was followed by adding chemical crosslinker N,N′-Methylenebisacrylamide (MBA) to generate crosslinks.

The rotation per minute was increased from 250 rpm to 500 rpm and MBA was allowed to dissolve for 15-20 min. The solution was then poured into 24 well plates and kept at 60° C. to set into gels. AMPS gel discs were collected as soon as the solution gelled and immediately transferred at 5° C. to stop further reaction.

For further experiments, these gels were swollen further to 500% of their original weight and cut into smaller softer discs using a glass test tube with diameter 1.5 cm. The hydrogels can be moulded into any shape depending upon the container in which the solution is poured.

TABLE 2
Composition of synthesized AMPS hydrogels

| Composition | Role | Molecular structure | Mol % |
|---|---|---|---|
| 2-Acrylamido-2- methylpropane sulfonic acid (AMPS-H) | Monomer | 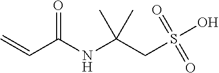 | 40-50% |
| Sodium | Neutraliser/ | NaOH | Equimolar ratio |
| hydroxide | metal complex | | to AMPS-H |
| N,N′-Methylene bis acrylamide (MBA) | Crosslinker | 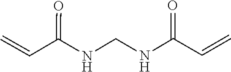 | 0.1-0.5% mol relative to AMPS-H |
| Potassium | Initiator | K₂S₂O₈ | 0.25-0.5% mol |
| persulfate (KPS) | | | relative to |
| | | | AMPS-H |

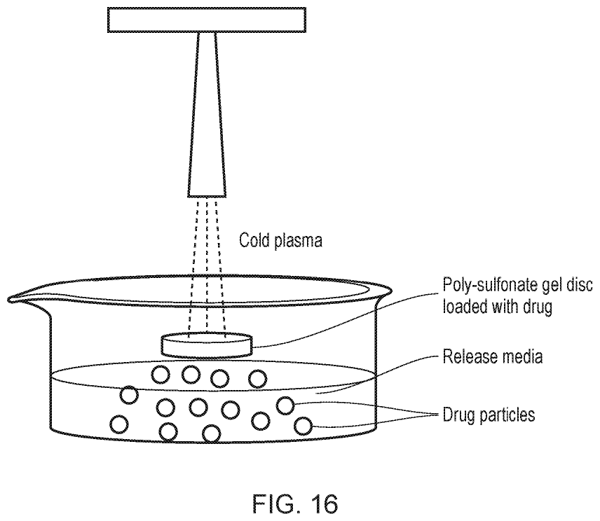

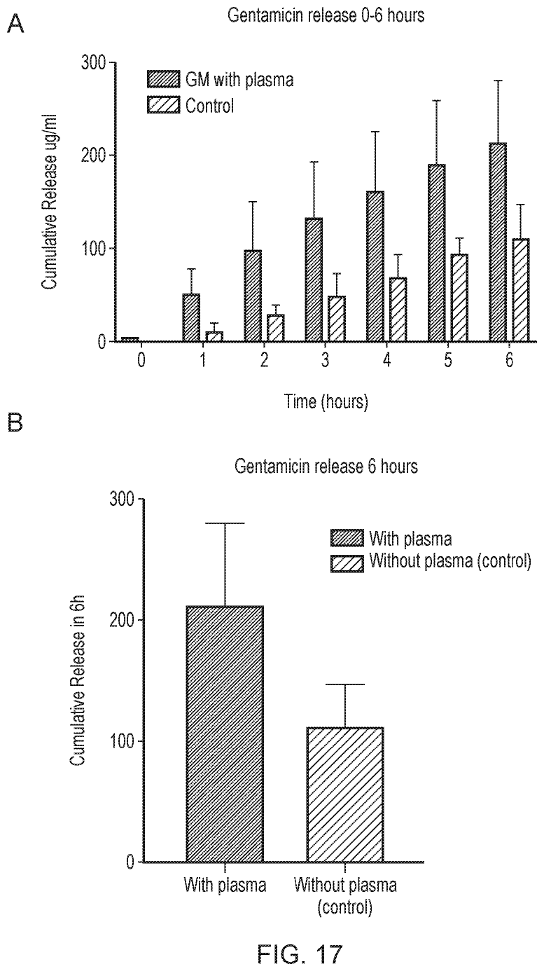

An AMPS hydrogel was loaded with gentamicin and subsequently treated with 4 minutes cold plasma to test gentamicin release as shown in FIG. 16. Gentamicin cumulative and total release are shown in FIGS. 17A and 17B. Gentamicin concentration was calculated using the Ninhydrin analytical assay. It can be seen that the plasma jet caused at least twice the amount of gentamicin to be released against control, where plasma was not applied at every time pint measured up to 6 hours.

1. A composite hydrogel comprising:
(a) an anionic or cationic functional group-containing polymer; and
(b) at least one active pharmaceutical ingredient (API), wherein the API is oppositely charged to the anionic or cationic functional group-containing polymer.

2. The composite hydrogel of claim 1, wherein the anionic functional group comprises at least one carboxylate functional group, or wherein the cationic functional group comprises at least one amino functional group.

3. The composite hydrogel of claim 1, wherein the anionic functional group-containing polymer is selected from one or more of poly(acrylic acid) (PAA) or poly(acrylic acid) partial sodium salt-graft-poly(ethylene oxide), poly X styrene sulphonic acid, poly X maleic anhydrides, 2-Acrylamido-2-methylpropane sulfonic acid or combinations thereof, or wherein the cationic functional group containing polymer is selected from one or more of poly-amino acids, polyamides and polymers grafted with amine containing side chains, cationic chitosan, cationic gelatin, cationic dextran, cationic cellulose, cationic cyclodextrin, Polyethyleneimine (PEI), Poly-_{L}-lysine (PLL), Poly(amidoamine) (PAA), Poly(amino-co-ester) (PAE), Poly(2-N,N-dimethylaminoethylmethacrylate) (PDMAEMA) or combinations thereof.

4. The composite hydrogel of claim 1, wherein the active pharmaceutical ingredient is an antimicrobial agent.

5. The composite hydrogel of claim 4, wherein the antimicrobial agent is selected from one or more of an antibacterial agent, an anti-biofilm agent or an anti-fungal agent.

6. The composite hydrogel of claim 1, wherein the active pharmaceutical ingredient is selected from one or more of silver ions, quaternary ammonium cations (QACs), amino glycoside antibiotics, N-acyl homo-serine lactones (AHLs), anti-microbial peptides (AMPs), Polymyxin antibiotics, enzymes, beta-lactam antibiotics, and/or combinations thereof.

7. The composite hydrogel of claim 6, wherein the quaternary ammonium cation (QAC) is cetrimide, and/or the amino glycoside antibiotic is gentamicin or streptomycin and/or the antimicrobial peptide (AMP) is a defensin and/or the Polymyxin antibiotic is polymyxin B and/or the beta-lactam antibiotic is amoxicillin.

8. The composite hydrogel of claim 1, wherein the active pharmaceutical ingredient is selected from, an anti-cancer agent, an anti-inflammatory agent, a tissue regenerative Active Pharmaceutical Ingredient (API), a gene therapy, an antibody and/or combinations thereof.

9. The composite hydrogel of claim 1, wherein the anionic or cationic functional group-containing polymer and the at least one active pharmaceutical ingredient are bound together to form an anionic or cationic functional group-containing polymer-pharmaceutically active agent complex.

10. The composite hydrogel of claim 9, wherein the anionic or cationic functional group-containing polymer-active pharmaceutical ingredient complex is bound by Coulombic interaction.

11. The composite hydrogel of claim 9, wherein the anionic or cationic functional group-containing polymer-active pharmaceutical ingredient complex is dispersed in the composite hydrogel.

12. The composite hydrogel of claim 1, wherein the hydrogel comprises a natural polymer or a synthetic polymer.

13. The composite hydrogel of claim 12, wherein the natural polymer is selected from one or more of hyaluronic acid, chitosan, heparin, alginate, and fibrin.

14. The composite hydrogel of claim 12, wherein the synthetic polymer is selected from one or more of polyvinyl alcohol, polyethylene glycol, sodium polyacrylate, acrylate polymers and copolymers thereof.

15. The composite hydrogel of claim 1, further comprising a diagnostic agent, wherein the diagnostic agent produces a visible signal in response to an infection.

16. The composite hydrogel of claim 14, wherein the diagnostic agent is selected from one or more of a dye or a pH indicator.

17. A method of displacing an active pharmaceutical ingredient from a composite hydrogel of claim 1, the method comprising a step of treating the composite hydrogel with an ion source comprising anions, cations, or a combination thereof.

18. The method of claim 17, wherein the ion source is selected from cold atmospheric plasma, plasma activated water, plasma or dielectric barrier discharge, glow discharge or an acid.

19. The method of claim 18, wherein the cold atmospheric plasma is a gas selected from an inert gas or a combination of an inert gas with air, oxygen and/or water vapour.

20. The method of claim 19, wherein the cold atmospheric plasma is an inert gas selected from one or more of argon, nitrogen or helium.

21. The method of claim 18, wherein the cold atmospheric plasma is excited and sustained by the application of radio frequency or microwave electrical power.

22. The method of claim 18, wherein the temperature of the cold atmospheric plasma is below about 45° C..

23. The method of claim 17, wherein the ion source is applied to the composite hydrogel for a period of from about 1 minute to about 10 minutes.

24. A wound dressing comprising the composite hydrogel of claim 1.

25. (canceled)

26. A method of treating a wound comprising the steps of:
(a) applying the composite hydrogel of claim 1, and
(b) displacing the at least one active pharmaceutical ingredient.
